(19) United States
(12) Patent Application Publication
(10) Pub. No.: US 2012/0303015 A1
(43) Pub. Date: Nov. 29, 2012

(54) ELECTRODE NEEDLE AND HEMOSTATIC DEVICE INCLUDING THE SAME

(75) Inventors: Kyung-Min Shin, Seoul (KR); Min-Woo Lee, Seoul (KR); Hyun-Chul Rhim, Seoul (KR)

(73) Assignees: Kyung-Min Shin, Seoul (KR); SAMSUNG LIFE PUBLIC WELFARE FOUNDATION, Seoul (KR); TAEWOONG MEDICAL CO., LTD., Gimpo, Gyeonggi-Do (KR)

(21) Appl. No.: 13/575,398

(22) PCT Filed: Jan. 24, 2011
(86) PCT No.: PCT/KR2011/000474

§ 371 Date: Jul. 26, 2012

(30) Foreign Application Priority Data
Jan. 26, 2010 (KR) 10-2010-0006997

(51) Int. Cl. A61B 18/18

(52) U.S. Cl. 606/33

(57) ABSTRACT

An electrode needle includes an electrode needle body, a metal tube, and an outer insulating tube. The electrode needle body is connected to a positive electrode of an RF generator. The metal tube is fixed around the electrode needle body and insulated from the electrode needle body. The metal tube is connected to a negative electrode of the RF generator. The outer insulating tube surrounds an upper part of the metal tube to expose a lower part of the metal tube. The outer insulating tube is movable longitudinally along the electrode needle body to vary an exposed area of the metal tube. At least one of a position, an area, and a cauterization speed of a site to be cauterized is controlled by varying the exposed area of the metal tube.

### TECHNICAL FIELD

The present invention relates to an electrode needle for radio frequency (RF) (including high frequency and microwave) ablation or hemostasis and a hemostatic device including the electrode needle, and more particularly, to an electrode needle configured to cauterize a lesion from which tissue is extracted for a biopsy or a bleeding site on an organ by RF heating, so as to stop bleeding and facilitate the regeneration of tissue, and a hemostatic device including the electrode needle.

### BACKGROUND ART

Generally, biopsies are performed to extract tissue from a lesion and examine the tissue, for example, by using a microscope.

Unlike cytodiagnosis, biopsies may be performed on any part of the body.

In recent needle biopsy, pathologic tissue to be examined is extracted from an organ such as the liver or kidney by using a needle. Usually, the extracted pathologic tissue is fixed in a 10% formalin solution and is embedded in paraffin. Then, the tissue is cut into thin pieces and stained with hematoxylin-eosin for microscope examination.

Final diagnoses of diseases such as tuberculosis and hepatitis may be made through histopathological examinations. Particularly, exact diagnoses of malignant tumors such as a cancerous tumor or a sarcoma are not possible without histopathological examinations.

The results of a biopsy are a diagnosis, although the results of a clinical examination may be data. Therefore, biopsies are carried out by pathologists.

Although the terms biopsy, tissue diagnosis, and histopathological examination have similar meanings, they are different. The term biopsy is used to denote the examination of a pathological sample taken from a living patient; and the terms tissue diagnosis and histopathological examination are used to denote the examination of a sample taken from a dissected human body or an animal, as well as from a living patient.

However, when tissue is extracted from a lesion for a biopsy, a site around the extracted tissue suffers from bleeding, and if the bleeding continues, the inside of the body may be contaminated by blood.

In addition, if bleeding continues, the site around the extracted tissue may be contaminated and infected, and cell regeneration may be lowered.

Therefore, there is a need for a hemostatic device configured to stop bleeding from a lesion from which tissue is extracted for a biopsy and facilitate the regeneration of tissue.

Furthermore, there is a need for an electrode needle for the cauterization, the area of which can be adjusted in an RF ablation treatment or a hemostatic treatment.

### DISCLOSURE OF INVENTION

### Technical Problem

An aspect of the present invention provides an electrode needle configured to perform cauterization while controlling at least one of a cauterization position, area, and speed, and a hemostatic device including the electrode needle.

Another aspect of the present invention provides an electrode needle configured to stop bleeding from a lesion from which tissue is extracted for a biopsy or bleeding from a site on an organ, and a hemostatic device including the electrode needle.

Another aspect of the present invention provides an electrode needle and a hemostatic device including the electrode needle, which are configured to cauterize a site around extracted pathological tissue by using RF heat, for minimizing or stopping bleeding and preventing secondary bodily contamination.

Another aspect of the present invention provides an electrode needle and a hemostatic device including the electrode needle, which are configured to facilitate regeneration of cells by cauterizing a site around extracted tissue through RF heating.

### Solution to Problem

According to an aspect of the present invention, there is provided an electrode needle including: an electrode needle body connected to a positive electrode of an RF (radio frequency) generator; a metal tube fixed around the electrode needle body and insulated from the electrode needle body, the metal tube being connected to a negative electrode of the RF generator; and an outer insulating tube surrounding an upper part of the metal tube to expose a lower part of the metal tube, the outer insulating tube being movable in a length direction of the electrode needle body to vary an exposed area of the metal tube, wherein at least one of a position, an area, and a cauterization speed of a site to be cauterized by an electrode needle tip portion of the electrode needle body and the exposed area of the metal tube is controlled by varying the exposed area of the metal tube.

The electrode needle may further include: an insulating tube surrounding the electrode needle body except for the electrode needle tip portion of the electrode needle body to insulate the electrode needle body and the metal tube from each other; and a ground insulating tube disposed around a part of the insulating tube between the electrode needle tip portion and the metal tube to insulate the electrode needle tip portion and the metal tube from each other.

The electrode needle may further include a fixing insulating tube making contact with an upper end of the metal tube and surrounding an outer surface of the insulating tube to prevent movement of the metal tube and the insulating tube.

The electrode needle may further include a temperature sensor inserted in the electrode needle body and connected to the RF generator.

According to another aspect of the present invention, there is provided a hemostatic device for stopping bleeding from a site from which tissue is extracted by using a biopsy needle or bleeding from a site of an organ, the hemostatic device including: a sheath configured to be placed at a lesion for a biopsy; and an electrode needle configured to be inserted into the sheath after a biopsy needle used to extract tissue from the lesion is detached from the sheath, so as to stop bleeding from the lesion from which the tissue is extracted or bleeding from a site of an organ, wherein the electrode needle includes: an electrode needle body connected to a positive electrode of an RF generator; a metal tube fixed around the electrode needle body and insulated from the electrode needle body, the metal tube being connected to a negative electrode of the RF generator; and an outer insulating tube surrounding an upper part of the metal tube to expose a lower part of the metal tube, the outer insulating tube being movable in a length direction of the electrode needle body to vary an exposed area of the metal tube.

### Advantageous Effects of Invention

According to the electrode needle and the hemostatic device of the present invention, continuous bleeding from a lesion from which tissue is extracted for a biopsy or continuous bleeding from a site of an organ can be prevented.

In addition, according to the present invention, at least one of the position, the area, and the cauterization speed of a site that will be cauterized by the electrode needle tip portion of the electrode needle body and the exposed region of the metal tube can be controlled by varying the exposed area of the metal tube. Therefore, the electrode needle and the hemostatic device can be used for various purposes such as hemostasis and RF ablation. In addition, the electrode needle and the hemostatic device can be flexibly used according to operational conditions.

In addition, according to the present invention, a site around extracted pathological tissue can be cauterized by RF heating for minimizing or stopping bleeding and preventing secondary bodily contamination.

Moreover, according to the present invention, regeneration of cells can be facilitated by cauterizing a site around extracted tissue through RF heating.

### BRIEF DESCRIPTION OF DRAWINGS

The above and other aspects, features and other advantages of the present invention will be more clearly understood from the following detailed description taken in conjunction with the accompanying drawings, in which:

FIG. 1 is a perspective view illustrating a hemostatic electrode needle for the bleeding site of an organ or the bleeding tissue after biopsy extraction, according to an embodiment of the present invention;

FIG. 2 is an enlarged view illustrating portion A of FIG. 1 according to an embodiment of the present invention;

FIG. 3 is a sectional view illustrating the electrode needle of FIG. 1 according to an embodiment of the present invention; and

FIGS. 4A and 4B are views for explaining a hemostatic operation using the electrode needle for the bleeding site of an organ or the bleeding tissue after biopsy extraction, according to an embodiment of the present invention.

### MODE FOR THE INVENTION

Exemplary embodiments of the present invention will now be described in detail with reference to the accompanying drawings.

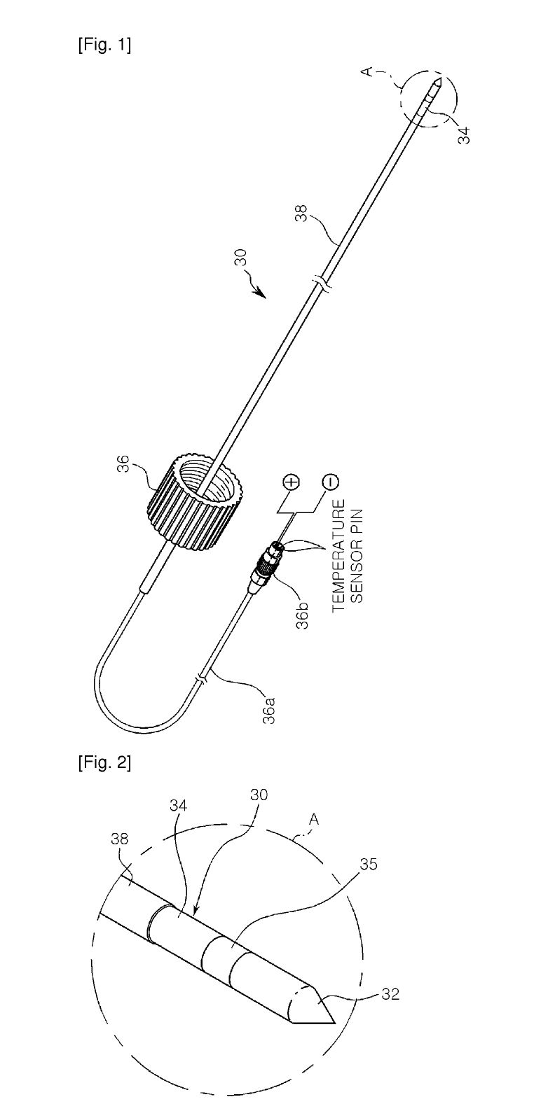

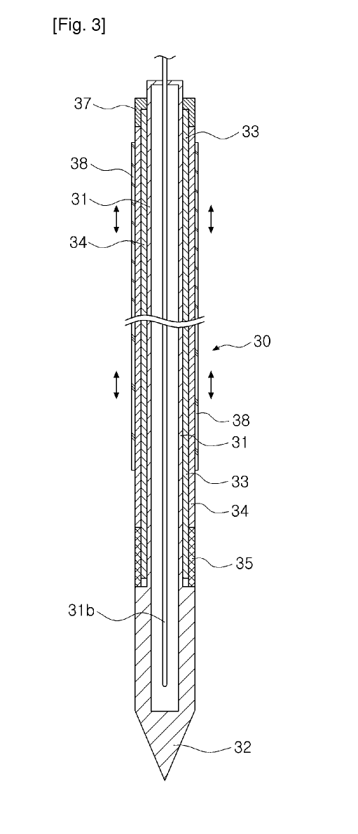

Referring to FIGS. 1 through 4, embodiments of the present invention provide an electrode needle 30 and a hemostatic device including the electrode needle 30. In a biopsy, a sheath 10 is placed on a bodily site, and tissue is extracted by using a biopsy needle 20. Then, the electrode needle 30 and the hemostatic device of the invention may be used to stop bleeding from a site around the extracted tissue or bleeding from a site of an organ by inserting the electrode needle 30 into the sheath 10.

In the following description, an explanation will be given of an exemplary case in which the electrode needle 30 and the hemostatic device are used to stop bleeding from a site around extracted tissue after a biopsy. However, the electrode needle 30 may be used for other purposes such as radio frequency (RF) ablation.

According to an embodiment of the present invention, the hemostatic device may be used to stop bleeding from a bodily site such as a site around extracted tissue. The hemostatic device includes the sheath 10 and the electrode needle 30. The sheath 10 is configured to be placed on a bodily site for a biopsy. After extracting tissue by inserting the biopsy needle 20, the biopsy needle 20 may be removed from the sheath 10, and the electrode needle 30 may be inserted into the sheath 10 to stop bleeding from a site around the extracted tissue or bleeding from a site of an organ.

First, the electrode needle 30 will be described according to an embodiment of the present invention. In the current embodiment, the electrode needle 30 is used for hemostasis. However, in other embodiments, the electrode needle 30 may be used for RF ablation to cauterize a lesion.

The electrode needle 30 of the current embodiment includes an electrode needle body 31 configured to be connected to a positive electrode of an RF generator (not shown), and a metal tube 34 insulated from the electrode needle body 31 and disposed around the electrode needle body 31. A negative electrode of the RF generator is connected to the metal tube 34. The metal tube 34 is fixed around the electrode needle body 31, and an insulating tube 33 is disposed between the metal tube 34 and the electrode needle body 31 to insulate the metal tube 34 from the electrode needle body 31.

In the current embodiment, the electrode needle 30 includes a ground insulating tube 35 and the insulating tube 33 for insulation between the electrode needle body 31 and the metal tube 34.

The insulating tube 33 surrounds the electrode needle body 31 except for an electrode needle tip portion 32 so that the outer surface of the electrode needle body 31 connected to the positive electrode of the RF generator can be insulated from the metal tube 34.

Thus, when RF waves (including high frequency waves and microwave) are transmitted to the electrode needle tip portion 32 to cauterize a lesion, other tissue may not be cauterized owing to the insulating tube 33.

The ground insulating tube 35 is fitted around a part of the insulating tube 33 between the electrode needle tip portion 32 and the metal tube 34 to insulate the electrode needle tip portion 32 and the metal tube 34 from each other.

A temperature sensor 3 lb is inserted in the electrode needle body 31 so that temperature can be monitored from the RF generator when a bleeding site is cauterized by using RF waves.

The electrode needle 30 is coupled to the front side of a main body 36 for easy handling. The electrode needle 30 receives RF waves from the RF generator (not shown), and an electrode line 36a connected to the temperature sensor 31b is coupled to the electrode needle body 31.

A female screw thread is formed on the inner surface of the main body 36 so that the main body 36 can be coupled to a body 11 of the sheath 10. During cauterization, the main body 36 can be used as a handle to control the electrode needle 30, and after cauterization, the main body 36 can be detached from the sheath 10.

An end of the electrode line 36a is connected to the temperature sensor 31b, and the electrode needle body 31 and the metal tube 34 that are coupled to the main body 36. The other end of the electrode line 36a is connected to a connector 36b connected to the RF generator. A positive electrode pin, a negative electrode pin, and a pair of temperature sensor pins connected to the temperature sensor 31b are connected from the connector 36b to the RF generator.

The electrode needle tip portion 32 of the electrode needle 30 is sharp, and the electrode needle body 31 has a diameter smaller than that of the electrode needle tip portion 32. The electrode needle body 31 is hollow so that the temperature sensor 31b can be inserted into the electrode needle body 31.

Since the diameter of the electrode needle body 31 is smaller than the diameter of the electrode needle tip portion 32, the insulating tube 33 or the metal tube 34 coupled to the electrode needle body 31 does not protrude from the electrode needle tip portion 32. Therefore, the electrode needle 30 may be smoothly inserted into a site from which tissue has been extracted.

In addition, the electrode needle 30 includes a fixing insulating tube 37 which is in tight contact with an end of the metal tube 34 and surrounds the outer surface of the insulating tube 33 to tightly hold the insulating tube 33 and the metal tube 34.

That is, the fixing insulating tube 37 is used to prevent a relative movement of the metal tube 34 when a cauterizing operation is performed.

In addition, the electrode needle 30 includes an outer insulating tube 38 surrounding an upper part of the metal tube 34 so that a lower part of the metal tube 34 can be exposed. The outer insulating tube 38 can be slid back and forth in the length direction of the electrode needle body 31. Therefore, the depth, position and size of a site to be cauterized can be controlled by adjusting the exposed area of the metal tube 34 with the outer insulating tube 38.

In the electrode needle 30 of the current embodiment, at least one of the position, area, cauterization speed of a site that will be cauterized by the electrode needle tip portion 32 of the electrode needle body 31 and the exposed region of the metal tube 34 can be controlled by varying the exposed area of the metal tube 34 using the outer insulating tube 38.

In detail, RF waves of 100 kilohertz to several megahertz are generally used for hemostasis or RF ablation. Frictional heat is generated in cells by applying AC energy to the cells through the electrode needle 30, and then the cells are solidified due to heating.

In the electrode needle 30 of the current embodiment, the exposed area of the metal tube 34 can be adjusted. For example, if the exposed area of the metal tube 34 is equal to the area of the electrode needle tip portion 32, cauterization occurs by both the exposed region of the metal tube 34 and the electrode needle tip portion 32. On the other hand, if the exposed area of the metal tube 34 is not equal to the area of the electrode needle tip portion 32, cauterization occurs mainly by one of the exposed region of the metal tube 34 and the electrode needle tip portion 32 which has a smaller area. Therefore, a cauterization area can be controlled by varying the exposed area of the metal tube 34 connected to the negative electrode with respect to the area of the electrode needle tip portion 32 connected to the positive electrode. In addition, it is possible to perform cauterization by using only one of them.

Furthermore, the time and/or speed of cauterization can be controlled by varying the exposed area of the metal tube 34. That is, since cauterization grows in intensity as the exposed area of the metal tube 34 is reduced, the speed of cauterization can be controlled by varying the exposed area of the metal tube 34. Thus, the time of cauterization can be controlled.

An exemplary use and effects of the electrode needle and the hemostatic device will now be described.

As shown in FIGS. 1 through 4, for a biopsy, the sheath 10 is inserted to a lesion in a manner such that the frontal end of the sheath 10 can be close to the lesion.

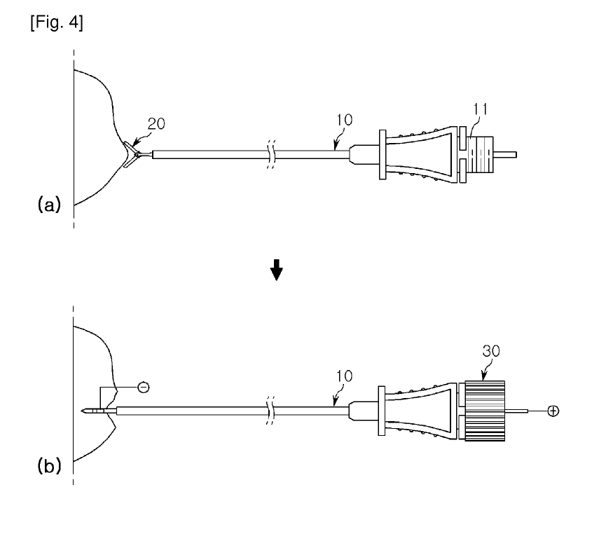

Next, the biopsy needle 20 is inserted into the sheath 10 (refer to FIG. 4A), and a part of the lesion is extracted by using the biopsy needle 20. Then, the biopsy needle 20 is detached from the sheath 10 but the sheath 10 is not removed.

Next, the electrode needle 30 is connected to an RF generator (not shown) which is a kind of RF ablation device by connecting the connector 36b connected to the electrode line 36a to the RF generator.

Thereafter, the electrode needle body 31 of the electrode needle 30 is inserted into the sheath 10 until the electrode needle tip portion 32 and the metal tube 34 are brought into contact with the lesion a part which has been extracted (refer to FIG. 4B).

Next, the RF generator is operated to generate RF waves, and then heat is generated around the electrode needle body 31 connected to a positive electrode and the metal tube 34 connected to a negative electrode, and thus the lesion a part of which has been extracted can be cauterized.

At this time, since the outer surface of the electrode needle body 31 is insulated by the insulating tube 33 surrounding the electrode needle body 31, RF waves may not affect normal tissue.

In addition, the exposed area of the metal tube 34 can be adjusted by moving the outer insulating tube 38 in the length direction of the electrode needle body 31, so as to control at least one of the position and size of a site that will be cauterized by the electrode needle tip portion 32 and the exposed region of the metal tube 34, and the speed of the cauterization.

After the cauterization, the sheath 10 and the electrode needle 30 are detached from the lesion.

In this way, bleeding from the lesion from which tissue is extracted can be rapidly stopped by cauterizing the lesion using RF waves, and thus contamination and infection of the lesion or other parts of the body can be prevented.

In addition to this, regeneration of tissue may be facilitated.

In the above embodiment, the electrode needle 30 is used after a biopsy. However, the electrode needle 30 may also be used for other purposes. For example, the electrode needle 30 may be directly inserted into a bleeding site of an organ without using the sheath 10 to cauterize the bleeding site. That is, the case shown in FIGS. 4A and 4B is merely an example of the present invention.

In addition, the electrode needle 30 may be used for RF ablation as well as hemostasis. For example, the electrode needle 30 may be used to cauterize a lesion.

While the present invention has been shown and described in connection with the exemplary embodiments, it will be apparent to those skilled in the art that modifications and variations can be made without departing from the spirit and scope of the invention as defined by the appended claims.

1. An electrode needle comprising:
an electrode needle body connected to a positive electrode of an RF (radio frequency) generator;
a metal tube fixed around the electrode needle body and insulated from the electrode needle body, the metal tube being connected to a negative electrode of the RF generator; and
an outer insulating tube surrounding an upper part of the metal tube to expose a lower part of the metal tube, the outer insulating tube being movable in a length direction of the electrode needle body to vary an exposed area of the metal tube,
wherein at least one of a position, an area, and a cauterization speed of a site to be cauterized by an electrode needle tip portion of the electrode needle body and the exposed area of the metal tube is controlled by varying the exposed area of the metal tube.

2. The electrode needle of claim 1, further comprising:
an insulating tube surrounding the electrode needle body except for the electrode needle tip portion of the electrode needle body to insulate the electrode needle body and the metal tube from each other; and
a ground insulating tube disposed around a part of the insulating tube between the electrode needle tip portion and the metal tube to insulate the electrode needle tip portion and the metal tube from each other.

3. The electrode needle of claim 2, further comprising a fixing insulating tube making contact with an upper end of the metal tube and surrounding an outer surface of the insulating tube to prevent movement of the metal tube and the insulating tube.

4. The electrode needle of claim 1, further comprising a temperature sensor inserted in the electrode needle body and connected to the RF generator.

5. A hemostatic device for stopping bleeding from a site from which tissue is extracted by using a biopsy needle or bleeding from a site of an organ, the hemostatic device comprising:
a sheath configured to be placed at a lesion for a biopsy; and
an electrode needle configured to be inserted into the sheath after a biopsy needle used to extract tissue from the lesion is detached from the sheath, so as to stop bleeding from the lesion from which the tissue is extracted or bleeding from a site of an organ,
wherein the electrode needle comprises:
an electrode needle body connected to a positive electrode of an RF generator;
a metal tube fixed around the electrode needle body and insulated from the electrode needle body, the metal tube being connected to a negative electrode of the RF generator; and
an outer insulating tube surrounding an upper part of the metal tube to expose a lower part of the metal tube, the outer insulating tube being movable in a length direction of the electrode needle body to vary an exposed area of the metal tube.

6. The hemostatic device of claim 5, wherein the electrode needle further comprises:
an insulating tube surrounding the electrode needle body except for an electrode needle tip portion of the electrode needle body to insulate the electrode needle body and the metal tube from each other; and
a ground insulating tube disposed around a part of the insulating tube between the electrode needle tip portion and the metal tube to insulate the electrode needle tip portion and the metal tube from each other.

7. The hemostatic device of claim 6, where the electrode needle further comprises a fixing insulating tube making contact with an upper end of the metal tube and surrounding an outer surface of the insulating tube to prevent movement of the metal tube and the insulating tube.

8. The hemostatic device of claim 5, wherein the electrode needle further comprises a temperature sensor inserted in the electrode needle body and connected to the RF generator, and the exposed area of the metal tube is adjusted based on a cauterization temperature measured through the temperature sensor
